(19) United States
(12) Patent Application Publication
(10) Pub. No.: US 2025/0011164 A1
(43) Pub. Date: Jan. 9, 2025

(54) SENSOR DEVICE

(71) Applicant: Infineon Technologies AG, Neubiberg (DE)

(72) Inventors: Klaus ELIAN, Alteglofsheim (DE); Jochen DANGELMAIER, Beratzhausen (DE); Rainer Markus SCHALLER, Aichen (DE)

(21) Appl. No.: 18/755,216

(22) Filed: Jun. 26, 2024

(30) Foreign Application Priority Data
Jul. 4, 2023 (DE) 102023206340.9

(51) Int. Cl. B81B 7/00; G01L 9/00; G01N 33/00

(52) U.S. Cl. CPC B81B 7/0087; G01L 9/00; G01N 33/0016; G01N 33/005; B81B 2201/0214; B81B 2201/0257; B81B 2207/012; B81B 2207/07; B81B 2207/092

(57) ABSTRACT

A sensor device includes a semiconductor chip and a package enclosing the semiconductor chip. The semiconductor chip includes integrated sensor circuitry configured to sense a characteristic of a gas in vicinity of the semiconductor chip. The package includes electrical contacts to the integrated sensor circuitry, at least one gas port enabling access of the gas into the package and to the semiconductor chip, a heating element configured to heat an interior portion of the package.

### CROSS-REFERENCE TO RELATED APPLICATION

This application claims priority to German Patent Application No. 102023206340.9 filed on Jul. 4, 2023, the content of which is incorporated by reference herein in its entirety.

### TECHNICAL FIELD

The implementation generally relates to sensor devices, in particular sensor devices for sensing a characteristic of gas.

### BACKGROUND

For sensing characteristics of gas, various kinds of semiconductor-based devices may be utilized. Such sensor devices typically include a semiconductor-chip adapted to sense a specific characteristic of gas in the environment of the semiconductor chip. For example, the semiconductor chip could be adapted to sense the presence and/or concentration of a certain type of gas.

To stabilize operation of such sensor device and/or to avoid adverse effects of humidity in the environment of the semiconductor chip, it is also known to provide the sensor device with an integrated heater. By way of example, US 2008/0134753 A1 describes a sensor device in which a gas-sensor chip structure also includes a heater.

Semiconductor chips for sensing characteristics of gases are typically tailored with respect to their specific purpose, e.g., the type of gas to be sensed, and adding an integrated heater in the chip structure may add complexity to design and manufacturing of the semiconductor chip.

Accordingly, there is a need for techniques which allow for efficiently providing gas sensor devices with heating capability. An object of the present disclosure is to accommodate such need.

### SUMMARY

The present disclosure provides a sensor device. The sensor device includes a semiconductor chip and a package enclosing the semiconductor chip. The semiconductor chip includes integrated sensor circuitry configured to sense a characteristic of a gas in vicinity of the semiconductor chip. The package enclosing the semiconductor chip includes electrical contacts to the integrated sensor circuitry, e.g., for interfacing the integrated sensor circuitry with a circuit board, such as a PCB, and at least one gas port enabling access of the gas into the package and to the semiconductor chip. Further, the package includes a heating element configured to heat an interior portion of the package.

### BRIEF DESCRIPTION OF THE DRAWINGS

Examples of the present disclosure will be described with reference to the following appended drawings, in which like reference signs refer to like elements.

FIG. 1 schematically illustrates an electronic system with a sensor device, according to an implementation of the present disclosure.

FIG. 2 schematically illustrates an example of a sensor device, according to an implementation of the present disclosure.

FIG. 3 schematically illustrates a further example of a sensor device, according to an implementation of the present disclosure.

FIG. 4 schematically illustrates a further example of a sensor device, according to an implementation of the present disclosure.

FIGS. 5A and 5B schematically illustrates a further example of a sensor device, according to an implementation of the present disclosure.

FIG. 6 schematically illustrates a further example of a sensor device, according to an implementation of the present disclosure.

FIG. 7 schematically illustrates a further example of a sensor device, according to an implementation of the present disclosure.

FIG. 8 schematically illustrates a further example of a sensor device, according to an implementation of the present disclosure.

FIG. 9 schematically illustrates a further example of a sensor device, according to an implementation of the present disclosure.

FIG. 10 schematically illustrates a further example of a sensor device, according to an implementation of the present disclosure.

FIG. 11 shows a flowchart for illustrating a method of manufacturing a sensor device, according to an implementation of the present disclosure.

It should be understood that the above-identified drawings are not intended to limit the scope of the present disclosure. Rather, these drawings are provided to assist in understanding the present disclosure. The person skilled in the art will readily understand that aspects of the present implementation shown in one drawing may be combined with aspects in another drawing or may be omitted without departing from the scope of the present disclosure.

### DETAILED DESCRIPTION

Concepts as illustrated in the present disclosure relate to sensor devices to be used for sensing one or more characteristics of one or more gases. Such sensor devices are herein also denoted as “gas sensor device”. The characteristic(s) to be sensed may for example correspond to presence and/or concentration of a certain gas type in the environment of the gas sensor device. For example, the gas sensor device could be used to detect the presence and/or concentration of hydrogen. However, other types of gas could be detected as well, e.g., carbon dioxide, carbon monoxide, oxygen, methane, mixtures of gases, or the like. Further, the gas sensor device could be used to detect pressure of the gas or variations of gas pressure. A specific case of a gas sensor device operating by detecting variations of gas pressure is a microphone.

In the illustrated concepts, the gas sensor device includes a semiconductor chip and a package enclosing the semiconductor chip. The semiconductor chip includes integrated sensor circuitry which configured to sense a characteristic of a gas in vicinity of the semiconductor chip. The specific configuration of the integrated circuitry and the semiconductor chip depends on the characteristic(s) and type(s) of gas to be sensed. In some cases, the semiconductor chip may include MEMS (Micro-Electro-Mechanical System) structures adapted to sense the characteristic(s).

The package enclosing the semiconductor chip provides electrical contacts to the integrated sensor circuitry, e.g., for interfacing the integrated sensor circuitry with a circuit board, such as a PCB. Further, the package provides at least one gas port enabling access of the gas to be sensed into the package and to the semiconductor chip. With such package, the gas sensor device can be mounted in various types of electronic systems, e.g., in a control system for a combustion engine or in a processing machine.

In practical use, gas sensor devices may however exhibit a cross-sensitivity with respect to other gases than that or those to be sensed. For example, a gas sensor device to be used for sensing the presence or concentration of hydrogen gas may also respond to the presence of humidity, e.g., water. Further, the response of a gas sensor device could also be temperature dependent. As a result, accuracy of measurement results provided by the gas sensor device could be adversely affected.

In the illustrated concepts, the package which is used to enclose the semiconductor chip is further provided with at least one heating element that is configured to heat an interior portion of the package. Due to the heating, other media than the gas(es) to be sensed can be at least in part be removed from the interior of the package and thus from the vicinity of the semiconductor chip. In this way, cross-sensitivity problems can be reduced. Further, the heating may be used to provide a stabilized environment for the sensing, thereby improving accuracy.

By integrating the heating element in the package, optimizing the design of the gas sensor device with respect to various applications can be facilitated. Specifically, the design of the semiconductor chip can be modified and tailored in various ways, without requiring modification of the package and heating element(s).

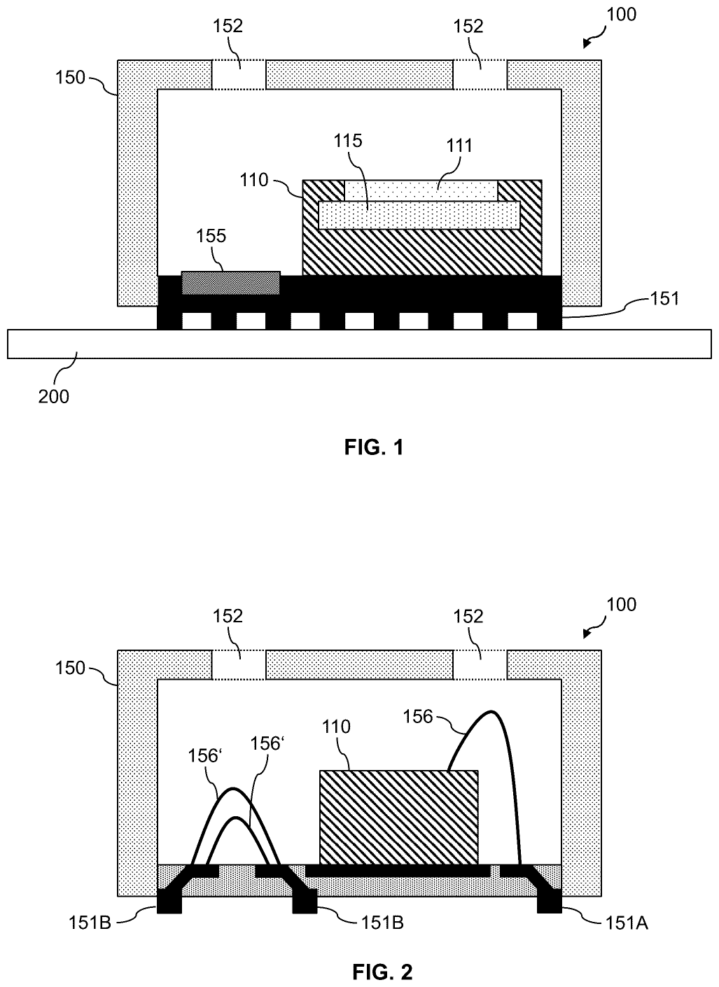

FIG. 1 schematically illustrates an example electronic system equipped with a gas sensor device 100 in accordance with the illustrated concepts. As illustrated, the gas sensor device 100 is mounted on a circuit board 200, e.g., a PCB. The circuit board 200 may connect the gas sensor device 100 to various other components of the electronic system.

As further illustrated, the gas sensor device 100 is based on a semiconductor chip 110 which is enclosed in a package 150. In the illustrated example, the semiconductor chip 110 is provided with a gas sensing region 111 that is exposed to gas in the vicinity of the semiconductor chip 110. The semiconductor chip 110 is adapted to sense one or more characteristics of the gas to which the gas sensing region 111 is exposed. Integrated circuitry 115 of the semiconductor chip 110 is configured to provide electric response signals which represent the characteristic(s) to be sensed. Here, it is noted that the specific configuration of the gas sensing region 111 and/or of the integrated circuitry 115 typically depend on the characteristic(s) and gas type(s) to be sensed and may be tailored with respect to the application the gas sensor 100 is intended for. In some cases, the gas sensing region 111 and the integrated circuitry may be MEMS based.

The package 150 encloses the semiconductor chip by casing material, e.g., plastic and/or ceramic, and provides an electric interface between the semiconductor chip 110 of the semiconductor chip 110 and the circuit board 200. More specifically, the package 150 may provide an electric interface between the integrated circuitry 115 of the semiconductor chip 110 and the circuit board 200 For this purpose, the package 150 is provided with a carrier 151 on which the semiconductor chip 110 is mounted, e.g., in the form of a leadframe. The carrier 151 may not only provide electrical contacts from the outside into the interior of the package 150, but also internal electrical interconnects, e.g., between different parts of the integrated circuitry 115 or between the semiconductor chip 110 and other components included in the package, e.g., a further semiconductor chip or one or more discrete circuit components. Further, the package is provided with at least one gas port 152 which allows gas from outside the package 150 to enter an interior portion of the package 150 so that the gas sensing region 111 of the semiconductor chip 110 can be exposed to the gas(es) to be sensed.

As further illustrated, a heating element 155 is integrated in the package 150. The heating element 155 allows for heating the interior portion of the package 150. In this way, other media than the gas(es) to be sensed, e.g., humidity, may be expelled from the package 150. Further, the temperature inside the package 150 may be stabilized. The heating element 155 may be electrically powered. In such case, electric power for the heating element 155 may be supplied from the circuit board 200, through the carrier 151. It is however noted that in some scenarios the heating element 155 could also operate as an inverse heat sink, using the contact structure 151 to conduct heat into the interior portion of the package 150. Further details concerning possible implementations of the heating element 155 will be explained below.

FIG. 2 schematically illustrates an example of a gas sensor device 100 in which the heating element 155 is in part implemented by the carrier 151. As mentioned above, the carrier 151 may correspond to a leadframe of the package and may provide electrical contacts with respect to the semiconductor chip 110. By way of example, FIG. 2 illustrates an electrical lead 151A which extends from outside into the package 150. A bond wire 156 provides an electrical connection of the electrical lead 151A to the semiconductor chip 110. It is noted for the sake of a better overview FIG. 2 illustrates only one electrical lead 151A for connecting to the semiconductor chip 110, but that in practical implementations there would typically be a plurality of such electrical leads 151A for connecting to the semiconductor chip 110.

As further illustrated, the carrier 151 also provides further electrical leads 151B which have the purpose of providing electrical energy to the heating element 155. In the example of FIG. 2, the heating element 155 is implemented based one or more bond wires 156′ connecting the electrical leads 151B. By supplying electric energy to the electrical leads 151B, electric current is injected to the bond wire(s) 156′ and in the leads 151B, thereby causing Ohmic heating.

With this structure, the heating element 155 may be implemented in a simple manner. Characteristics of the heating element 155 may be adapted by choice of the number of bond wires 156′, type of the bond wires and/or length of the bond wires 156′.

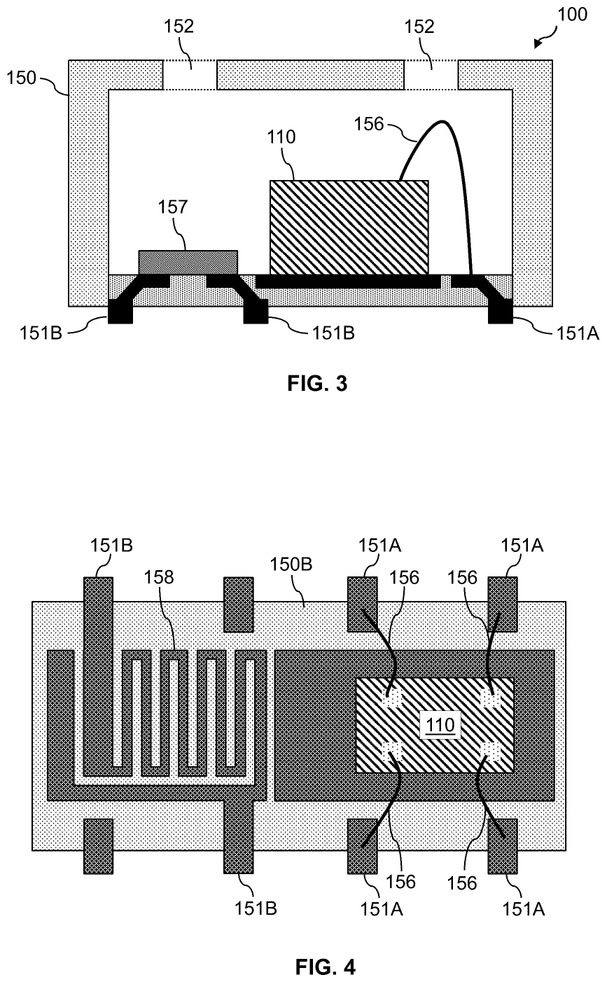

FIG. 3 schematically illustrates a further example of a gas sensor device 100. In the example of FIG. 3, the heating element 155 is implemented by a discrete resistor 156, e.g., an SMD (Surface Mounted Device) resistor. Otherwise, structures and functionalities of the gas sensor device 100 may be similar to those as explained in connection with FIGS. 1 and 2.

FIG. 4 schematically illustrates a further example of a gas sensor device 100. For the sake of a better overview, FIG. 4 shows a partial top view of the gas sensor device 100, including a bottom part 150B of the package 150 and the carrier 151, which is based on a patterned leadframe. In the example of FIG. 4, the heating element 155 is implemented by a patterned structure 158 of the leadframe. As can be seen, the patterned structure 158 connects electrical leads 151B. By supplying electrical energy to the contacts 151B, electric current is injected to the patterned structure 158 and into the leads 151B, thereby causing Ohmic heating. In the example of FIG. 4, the patterned structure 158 is provided with a meandering shape, which may help to tune its Ohmic resistance into a desirable range, without requiring excessive space. It is however noted that various other shapes are possible as well.

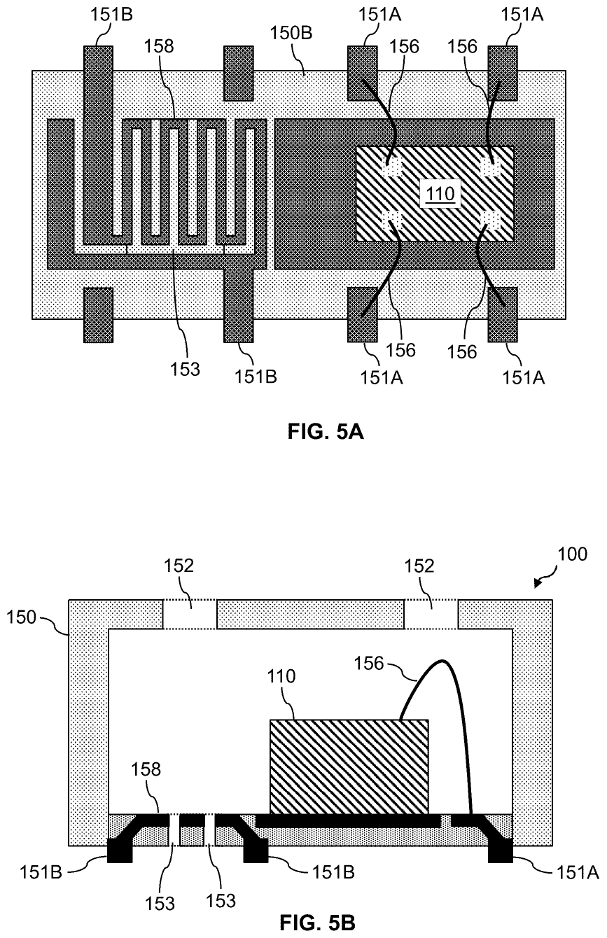

FIGS. 5A and 5B schematically illustrate a further example of a gas sensor device 100, which is generally similar to that of FIG. 4. However, as compared to the example of FIG. 4, in the example of FIGS. 5A and 5B the bottom part 150B of the package 150 includes one or more vent ports 153. These one or more vent ports 153 may be implemented by void areas of the patterned structure 158 of the leadframe, which are not covered by other package material, e.g., plastic or ceramic casing material. With respect to the gas ports 152, the vent ports 153 may be provided on an opposite side of the package 150. With such structure, it may be possible to enhance ventilation of the interior portion of the package 150, due to a stack effect.

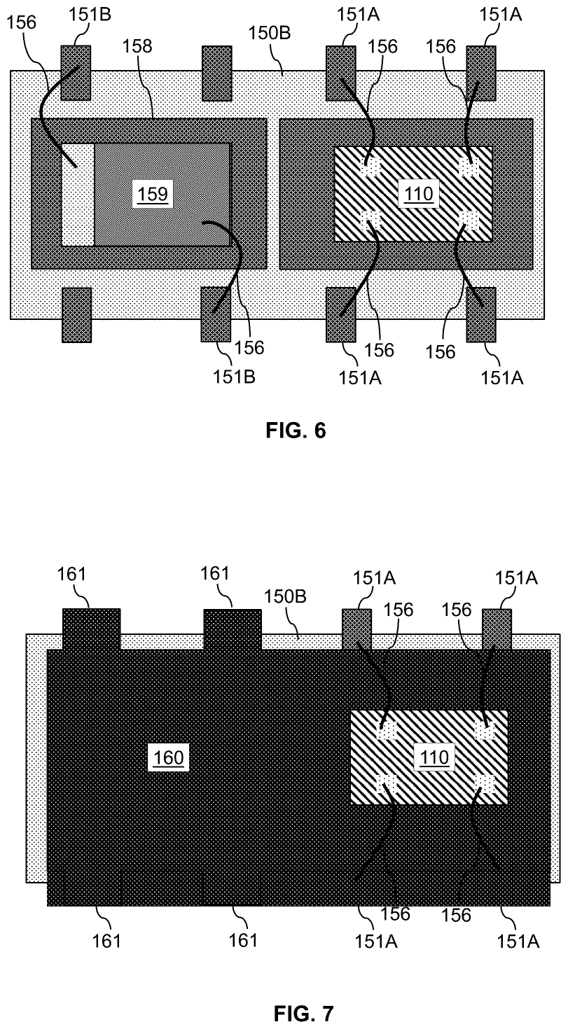

FIG. 6 schematically illustrates a further example of a gas sensor device 100. In the example of FIG. 6, the heating element 155 is implemented by a Peltier element 159. Such Peltier element 158 may be provided by a layer system embedded in the package 150, e.g., by adding additional layers to the leadframe forming the carrier 151. Alternatively, the Peltier element could also be provided in the form of a discrete component, similar to the SMD resistor in the example of FIG. 3. In the example of FIG. 6, the Peltier element 159 is formed by additional layers on the leadframe and connected to electrical leads 151B by bond wires 156. By supplying electric energy to the electrical leads 151B, the Peltier element 159 may provide heating due to the Peltier effect. It is however noted that the Peltier element 159 could in some scenarios also be used for cooling the interior portion of the package 150. The Peltier element 159 may thus be useful for stabilizing conditions in the interior portion of the package 150.

FIG. 7 schematically illustrates a further example of a gas sensor device 100. In the example of FIG. 7, the heating element 155 is implemented by a heat conductor 160 operating as an inverse heat sink. The heat conductor 160 may be formed of a metallic material. In the example of FIG. 7, the heat conductor 160 is assumed to be implemented as part of the carrier 151, e.g., as part of a leadframe. For example, the leadframe could be formed from dual-gauge material, using the higher gauge material to form the heat conductor 160. Further, metallic material forming the heat conductor 160 could be welded to the leadframe. Still further, the heat conductor 160 could be formed as part of a three-dimensional leadframe structure, enabling to form the heat conductor 160 on another level than structures for providing the electrical leads 151A.

As illustrated in FIG. 7, the heat conductor 160 is provided with thermal leads 161 which extend from outside the package 150 into the interior of the package 150. Outside the package 150, the thermal leads 161 may be connected to an external heat reservoir, e.g., on the circuit board. The external heat reservoir could draw heat from the environment or some external cooling system. In some cases, such external heat reservoir could also be actively heated by an external heater.

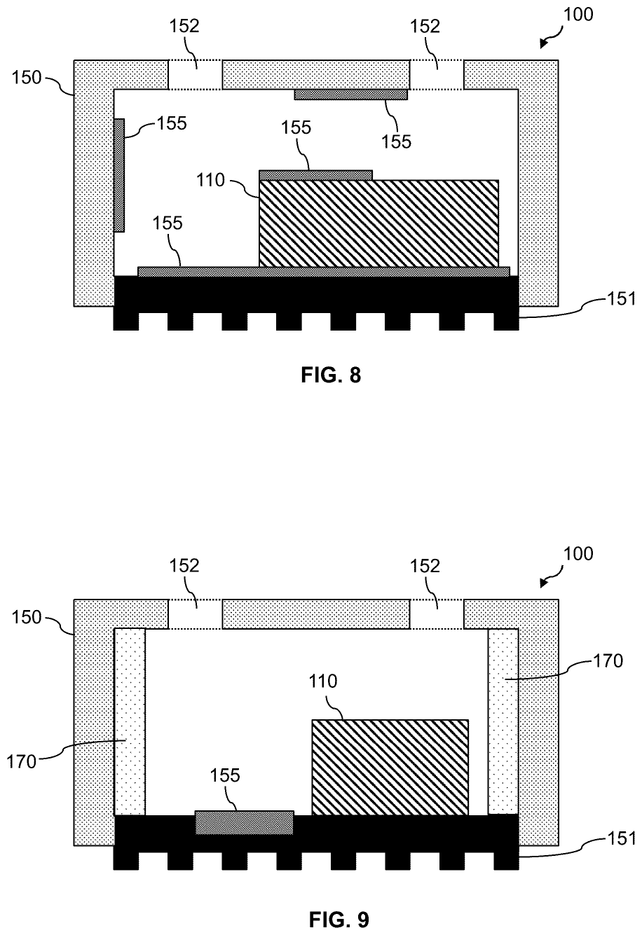

It is noted that the arrangement of the heating element 155 as illustrated in FIGS. 1 through 7 is merely an example and that other arrangements are possible. In some implementations, the heating element 155 may be side-by-side the semiconductor chip 110, e.g., like in the examples of FIGS. 1-6. In other implementations, the heating element 155 may be arranged underneath the semiconductor chip 110, e.g., as an additional layer on a carrier of the semiconductor chip 110. The arrangement of FIG. 7 is an example of such an implementation. Another example of such an implementation is illustrated in FIG. 8. As further illustrated in the example of FIG. 8, the heating element may be arranged as, or be part of, a cover of the semiconductor chip 110, on other interior surfaces of the package 150, or at any internal stack position within the package 150. Such arrangement of the heating element 155 may be achieved by implementing the heater element 155 based on a polyimide heating foil.

In some implementations, the gas sensor device 100 could also be provided with one or more insulating elements which provide heat insulation of the interior portion of the package 150. FIG. 9 schematically illustrates a corresponding example, where insulating elements 170 are provided on interior side surfaces of the package 150. Such heat insulating elements may be formed from a material of low heat conductance, e.g., a plastic material. By use of such insulating element, efficiency of the heating element 155 may be improved.

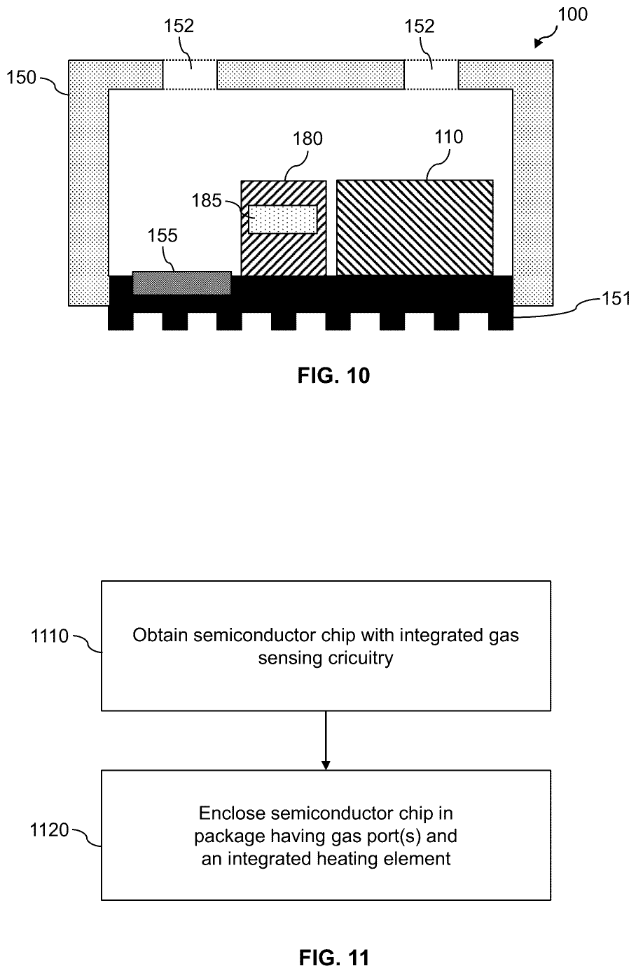

In some implementations, the heating element 155 could be operated in a controlled manner, e.g., by regulating the electrical power supplied to the heating element 155. In such cases, control circuitry for controlling the heating element 155 could also be integrated in the package 150. FIG. 10 schematically illustrates a corresponding example. In the example of FIG. 10, the package 150 includes, in addition to the semiconductor chip 110 adapted to perform the sensing, a further semiconductor chip 180 which is adapted to control the heating element 155. In such an implementation, feeding of the electric power to the heating element 155 may be done through control circuitry 185 integrated in the further semiconductor chip 180. Such control circuitry 185 could also include a temperature sensor for measuring the temperature in the interior portion of the package 150. Alternatively, such temperature sensor could also be by a discrete component within the package, e.g., a discrete resistor or a discrete diode.

It is noted that features of the gas sensor device 100 as explained in connection with FIGS. 1 through 10 may be combined in various ways. For example, different types of the illustrated heating element 155 could be combined within the same gas sensor device 100. For example, the heating element 155 could include a combination of two or more of: bond wires 156′ as illustrated in FIG. 2, a discrete resistor 157 as illustrated in FIG. 3, a patterned structure 158 of the leadframe as illustrated in FIGS. 4, 5A, and 5B, a Peltier element 159 as illustrated in FIG. 6, or a heat conductor 160 as illustrated in FIG. 7. Further, the one or vent ports 153 illustrated in the example of FIGS. 5A and 5B could also be provided in any of the examples of FIGS. 1 through 3 or 6 through 10.

FIG. 11 shows a flowchart for schematically illustrating manufacturing of a gas sensor device in accordance with the illustrated concepts. The method of FIG. 11 could for example be used for manufacturing the gas sensor device 100 of any of the above examples. The method of FIG. 11 could be performed by a device manufacturer. The method may be performed in an automated or semi-automated manner.

In the method of FIG. 11, block 1110 involves obtaining a semiconductor chip with integrated circuitry for sensing at least one characteristic of a gas. For example, block 110 could involve obtaining the semiconductor chip 110. The semiconductor chip may be obtained from a chip vendor or could be manufactured as initial part of block 1110.

At block 1120, the semiconductor chip obtained at block 1110 is packaged. This involves enclosing the semiconductor chip in a package. The package provides electrical contacts to the integrated sensor circuitry. Further, the package provides at least one gas port enabling access of the gas into the package and to the semiconductor chip. As a further element, the package includes a heating element configured to heat an interior portion of the package. The package may for example have structures as explained in connection with FIGS. 1 through 10.

As can be seen, by using the method of FIG. 11, a semiconductor chip for gas sensing may be supplemented with a heating capability, without requiring modification of the semiconductor chips design.

In view of the above, examples provided by the present disclosure include a sensor device which comprises a semiconductor chip and a package enclosing the semiconductor chip, where the semiconductor chip comprises integrated sensor circuitry configured to sense a characteristic of a gas in vicinity of the semiconductor chip, and where the package comprises electrical contacts to the integrated sensor circuitry, e.g., electrical contacts for contacting the integrated sensor circuitry to a circuit board, at least one gas port enabling access of the gas into the package and to the semiconductor chip; and a heating element configured to heat an interior portion of the package.

The package may comprise a carrier on which the semiconductor chip is mounted and which supports the electrical contacts, such as the above-mentioned carrier 151. At least a part of the heating element may be integrated in the carrier. This part can for example include leads to the heating element and/or the heating element itself, e.g., e lead structure.

The carrier may comprise a first set of leads for providing the electrical contacts to the sensor circuitry, such as the above-mentioned leads 151A, and a second set of leads for providing electrical energy to the heating element, such as the above-mentioned leads 151B. In such case, the heating element may comprise a further lead structure of the carrier, e.g., such as the above-mentioned patterned structure 158 of the leadframe. The further lead structure may be connected to the second set of leads and configured to provide Ohmic heating by a current flowing through the second set of leads. Alternatively, or in addition, the heating element could comprise bond wires connected to the second set of leads, such as the bond wires 156′ in the example of FIG. 2.

In some implementations, the package may comprise a cover arranged on the semiconductor chip. In such case, at least a part of the heating element could be integrated in the cover, e.g., as illustrated in the example of FIG. 8.

In some implementations, the heating element may be at least partially embedded in material of the package, e.g., as in the examples of FIGS. 1, 2, 3, 4, 5A, 5B, 6, 7, 9, and 10.

In some implementations, the heating element may comprise a heating resistor, e.g., like in the examples of FIGS. 2 and 8. In some case, such heating resistor could be based on a polyimide film.

In some implementations, the heating element may comprise a Peltier element, e.g., like in the example of FIG. 6.

In some implementations, the heating element may comprise a heat conductor configured to conduct heat from outside the package into the package, e.g., like in the example of FIG. 7.

In some implementations, the sensor device may further comprise at least one vent port in gas communication with the at least one gas port, e.g., like the vent ports 153 in the example of FIGS. 5A and 5B. In such case, the at least one gas port and the at least one vent port can be located on opposite sides of the package.

In some implementations, the sensor device may further comprise at least one insulating element configured to provide heat insulation of the interior portion of the package with respect to outside the package, such as the insulating elements in the example of FIG. 9.

In some implementations, the sensor device may further comprise a further semiconductor chip enclosed by the package, with the further semiconductor chip comprising control circuitry configured to control operation of the heating element, e.g., like the further semiconductor chip 180 in the example of FIG. 10.

In some implementations, the sensor circuitry may be is configured to detect presence of hydrogen in the gas. In some scenarios, the sensor circuitry may be configured to detect pressure of the gas or pressure variations of the gas. In some scenarios, the sensor device may be a microphone.

It is noted that the above-described concepts and examples are susceptible to various modifications. For example, the concepts could be applied to various sensor devices configured to sense types of gas characteristics, without limitation to the above-mentioned examples of characteristics. Accordingly, the illustrated concepts may be applied to various kinds and designs of semiconductor chips. Further, the concepts could be applied in connection with various types of package, e.g., dual-line packages, pin grid array packages, ball grid array packages, land grid array packages, or surface mount packages.

1. A sensor device, comprising:
a semiconductor chip comprising integrated sensor circuitry configured to sense a characteristic of a gas in a vicinity of the semiconductor chip; and
a package enclosing the semiconductor chip, the package comprising:
electrical contacts to the integrated sensor circuitry;
at least one gas port enabling access of the gas into the package and to the semiconductor chip; and
a heating element configured to heat an interior portion of the package.

2. The sensor device of claim 1, wherein the package comprises a carrier on which the semiconductor chip is mounted and which supports the electrical contacts, and
wherein at least a part of the heating element is integrated in the carrier.

3. The sensor device of claim 2, wherein the carrier comprises a first set of leads for providing the electrical contacts to the integrated sensor circuitry and a second set of leads for providing electrical energy to the heating element.

4. The sensor device of claim 3, wherein the heating element comprises a further lead structure of the carrier connected to the second set of leads and configured to provide Ohmic heating by a current flowing through the second set of leads.

5. The sensor device of claim 3, wherein the heating element comprises bond wires connected to the second set of leads.

6. The sensor device of claim 1, wherein the package comprises a cover arranged on the semiconductor chip, and
wherein at least a part of the heating element is integrated in the cover.

7. The sensor device of claim 1, wherein the heating element is at least partially embedded in a material of the package.

8. The sensor device of claim 1, wherein the heating element comprises a heating resistor.

9. The sensor device of claim 8, wherein the heating resistor is based on a polyimide film.

10. The sensor device of claim 1, wherein the heating element comprises a Peltier element.

11. The sensor device of claim 1, wherein the heating element comprises a heat conductor configured to conduct heat from outside the package into the package.

12. The sensor device of claim 1, further comprising:
at least one vent port in gas communication with the at least one gas port.

13. The sensor device of claim 12, wherein the at least one gas port and the at least one vent port are located on opposite sides of the package.

14. The sensor device of claim 1, further comprising:
at least one insulating element configured to provide heat insulation of the interior portion of the package with respect to outside the package.

15. The sensor device of claim 1, further comprising:
a further semiconductor chip enclosed by the package, wherein the further semiconductor chip comprises control circuitry configured to control operation of the heating element.

16. The sensor device of claim 1, wherein the integrated sensor circuitry is configured to detect presence of a hydrogen in the gas.

17. The sensor device of claim 1, wherein the integrated sensor circuitry is configured to detect a pressure of the gas or pressure variations of the gas.

18. The sensor device of claim 1, wherein the sensor device is a microphone.

19. A package for enclosing a semiconductor chip comprising integrated circuitry configured to sense a characteristic of a gas, the package comprising:
electrical contacts to the integrated circuitry;
a gas port configured to enable an access of the gas into the package and to the semiconductor chip; and
a heating element configured to heat an interior portion of the package.

20. The package of claim 19, further comprising:
a carrier configured to support the electrical contacts and to mount the semiconductor chip, wherein at least a portion of the heating element is integrated in the carrier.
